(19) United States
(12) Patent Application Publication
(10) Pub. No.: US 2020/0200775 A1
(43) Pub. Date: Jun. 25, 2020

(54) OXIDIZED LIPIDS AS BIOMARKERS FOR NEUROPATHIC PAIN

(71) Applicants: Fraunhofer-Gesellschaft zur Förderung der angewandten Forschung e.V., München (DE); Johann Wolfgang Goethe-Universität Frankfurt, Frankfurt am Main (DE)

(72) Inventors: Marco SISIGNANO, Frankfurt (DE); Gerd GEISSLINGER, Bad Soden (DE); Michael John PARNHAM, Bad Soden (DE)

(21) Appl. No.: 16/608,779

(22) PCT Filed: Apr. 26, 2017
(86) PCT No.: PCT/EP2017/059996

§ 371 Date: Oct. 25, 2019

(51) Int. Cl. G01N 33/92; G01N 35/00

(52) U.S. Cl. CPC G01N 33/92; G01N 35/00584; G01N 2800/52; G01N 2800/50; G01N 2405/00; G01N 2800/2842

(57) ABSTRACT

Diagnostic methods are useful for diagnosing neuropathic pain in a subject, for predicting whether a subject is at risk of developing neuropathic pain, or for determining whether a neuropathic pain therapy is successful. Tools for carrying out the aforementioned methods, include diagnostic devices, and oxidized lipids, for example, an epoxylipid, for use in the aforementioned methods.

The present invention relates to the field of diagnostic methods. Specifically, the present invention relates to a method for diagnosing neuropathic pain in a subject, a method for predicting whether a subject is at risk of developing neuropathic pain or a method for determining whether a neuropathic pain therapy is successful. The invention also relates to tools for carrying out the aforementioned methods, such as diagnostic devices and to an oxidized lipid, preferably an epoxylipid, for use in the aforementioned methods.

Neuropathic pain is a persistent or chronic pain syndrome that can result from damage to the nervous system, the peripheral nerves, the dorsal root ganglion, dorsal root, or to the central nervous system. Neuropathic pain syndromes include allodynia, various neuralgias such as post herpetic neuralgia and trigeminal neuralgia, phantom pain, and complex regional pain syndromes, such as reflex sympathetic dystrophy and causalgia. Causalgia is often characterized by spontaneous burning pain combined with hyperalgesia and allodynia. Tragically there is no existing method for adequately, predictably and specifically treating established neuropathic pain as present treatment methods for neuropathic pain consist of merely trying to help the patient cope through psychological or occupational therapy, rather than by reducing or eliminating the pain experienced. Treatment of neuropathic or chronic pain is a challenge for physicians and patients since there are no medications that specifically target the condition, and since the medications presently used result in only little relief and are based on their efficacy in acute pain conditions or on their efficacy on relieving secondary effects like anxiety and depression. Incidence of chronic pain is increasing in society and its burden on society is huge in both health care and lost productivity. Currently there are no scientifically validated therapies for relieving chronic pain. As a result, the health community targets ‘pain management’ where multi-modal therapies are used concurrently with the hope of providing some improvement in quality of life. Thus, there is an urgent need for drugs that can relieve chronic pain.

Chemotherapy-induced neuropathic pain, also referred to as chemotherapy-induced peripheral neuropathy (CIPN), is a severe dose limiting side effect of cytostatics, such as taxanes, platinum derivates, vinca alkaloids and others. The symptoms usually start with tingling and can lead to burning, stabbing and aching pain as well as cold and mechanical allodynia. Due to CIPN some patients stop anticancer therapy with cytostatics too early, resulting in a higher risk of tumor progression. Unfortunately, many promising substances that are already approved for the treatment of different kinds of neuropathic pain, such as gabapentin or amitriptyline seem to have little or no analgesic effect in monotherapy of CIPN. Understanding the cellular and molecular mechanisms is necessary to treat or even prevent CIPN and may improve the general success rate of cytostatic therapy.

Early therapeutic intervention is crucial for the therapy of neuropathic pain [1, 2]. For this reason, biomarkers are especially important for neuropathic pain and represent important diagnostic markers that may be used for therapeutic strategies. Particularly during treatment of patients with cytostatics or during diabetes, the onset and intensity of neuropathic pain varies strongly among patients. In the ideal case, biomarkers may be measured from plasma of patients and analyzed for their concentrations. This can be used to predict onset, intensity and duration of neuropathic pain even before the first symptoms arise in patients [3]. In this regard, high-risk patients could be treated preventatively with drugs that are effective for the treatment of neuropathic pain, such as amitriptyline, gabapentin or duloxetine as early as possibly to reduce or even prevent neuropathic pain.

Currently, there are no biomarkers available for the prediction of onset, intensity or duration of neuropathic pain in patients. However, there is a strong demand for such biomarkers because neuropathic pain is still difficult to treat and can persist lifelong, especially when it is already fully established. There are several studies concerning genetic causes [7], such as single nucleotide polymorphisms or punctual mutations, that may explain some pain syndromes (like the familial episodic pain syndrome [8]), however, to this day, there are no predictive biomarkers, that could be used to judge a person's susceptibility to developing neuropathic pain.

Various (oxidized) lipids or (oxidized) fatty acids have been related to different kinds of pain including neuropathic pain, however reliable biomarkers for diagnosing and/or predicting neuropathic pain are currently unknown.

Inceoglu et al. used a streptozocin(STZ)-induced type I diabetes rat model to show that inhibition of epoxide hydrolase (sEH-1) can reduce pain related behavior due to STZ treatment in these rats by modulating the ratio of epoxy to hydroxyl fatty acids [9].

WO2010/062900 [10] relates to the use of different compounds or pharmaceutical compositions for treating pain, shock and/or inflammatory conditions in a subject. Such a pharmaceutical composition may include a lipoxygenase inhibitor, a cytochrome P-450 enzyme inhibitor, an antibody that bind to oxidized linoleic acid metabolites and/or an antioxidant.

WO2009/062073 [11] is concerned with the alleviation of neuropathic pain with cis-epoxyeicosantrienoic acids (EETs) and inhibitors of soluble epoxide hydrolase (sEH).

Kunori et al. discloses that Prostaglandin E2 blocks microglial migration in the spinal cord. Mice deficient in microsomal prostaglandin. E synthase did not exhibit mechanical allodynia after peripheral nerve injury [12].

Ramsden et al. discloses that a dietary intervention increasing n-3 and reducing n-6 fatty acids was beneficial in reduced headache pain [13].

The technical problem underlying the present invention can be seen as the provision of means and methods for complying with the aforementioned needs. The technical problem is solved by the embodiments characterized in the claims and herein below.

The present invention pertains to a method for diagnosing neuropathic pain in a subject comprising the steps of

(a) determining in a plasma sample of a subject suspected to suffer from neuropathic pain the amount of at least one oxidized lipid;

(b) comparing the said amount of the at least one oxidized lipid with a reference amount whereby neuropathic pain is to be diagnosed.

The method as referred to in accordance with the present invention includes a method which essentially consists of the aforementioned steps or a method which includes further steps. However, it is to be understood that the method, in a preferred embodiment, is a method carried out ex vivo, i.e. not practised on the human or animal body. The method, preferably, can be assisted by automation.

The term “diagnosing” as used herein refers to assessing whether a subject suffers from neuropathic pain, or not. As will be understood by those skilled in the art, such an assessment, although preferred to be, may usually not be correct for 100% of the investigated subjects. However, the term “diagnosing” requires that a statistically significant portion of subjects can be correctly assessed and, thus, diagnosed. Whether a portion is statistically significant can be determined without further ado by the person skilled in the art using various well known statistic evaluation tools, e.g., determination of confidence intervals, p-value determination, Student's t-test, Annova, Mann-Whitney test, etc. Details can be found in Dowdy and Wearden, Statistics for Research, John Wiley & Sons, New York 1983. Preferred confidence intervals are at least 50%, at least 60%, at least 70%, at least 80%, at least 90%, at least 95%. The p-values are, preferably, 0.2, 0.1, 0.05, 0.01, 0.005, 0.001.

The term includes individual diagnosis of neuropathic pain or its symptoms as well as continuous monitoring of a patient. Monitoring refers to diagnosing the presence or absence of neuropathic pain or the symptoms accompanying it at various time points. Furthermore, monitoring can also be used to determine whether a patient is treated successfully or whether at least symptoms of neuropathic pain can be ameliorated over time by a certain therapy.

The term “neuropathic pain” as used herein relates to a disturbance of function, pathological change and/or damage of nerve cells, mainly affecting the somatosensory nerve cells, causing pain. Disorders and/or diseases leading to neuropathic pain as well as symptoms associated therewith are known to the person skilled in the art and include, for example, abnormal sensations (dysesthesia) or pain from normally non-painful stimuli (allodynia). It is understood by those skilled in the art that neuropathic pain may be divided into different categories such as peripheral neuropathic pain, central neuropathic pain, or mixed (peripheral and central) neuropathic pain. Neuropathic pain may be caused by various disorders or conditions. Preferably, neuropathic pain includes post-herpetic neuralgia, trigeminal neuralgia, focal peripheral nerve injury, and anesthesia dolorosa, central pain due to stroke or mass lesion, spinal cord injury, or multiple sclerosis, and peripheral neuropathy due to diabetes, HIV, or chemotherapy. Most preferably, neuropathic pain is chemotherapy-induced neuropathic pain (CIPN).

Peripheral neuropathic pain is typically seen as disturbance of function or pathological change in a sensory nerve causing pain. This may be mediated by a lesion or disease of the peripheral somatosensory nervous system and typically appears as pain in the extremities (feet or hands) caused by light mechanical stimulations (such as touch) or cold temperatures. However, peripheral neuropathic pain may as well appear without stimulations (spontaneous pain).

The term “Chemotherapy-induced neuropathic pain”, also referred to as “chemotherapy-induced peripheral neuropathy” or “CIPN”, relates to neuropathic pain that is that is induced by a chemotherapeutic agent (also known as cytotoxic or cytostatic agents). CIPN is known to be a side effect (adverse event) of cancer therapy and is caused by the toxicity of (certain) cancer therapeutics. CIPN is one of the major reasons for delay or discontinuation of chemotherapy and therefore responsible for decreased chemotherapeutic efficacy and loss of quality of life. Typical symptoms of CIPN include pain, tingling, numbness and temperature sensitivity. Sometimes, also motor nerves/central nervous system and/or the autonomic nervous system are affected. In general, the nerve endings in the extremities of the hands and feet are affected earliest by toxicity in a symmetrical, length-dependent manner. Large sensory nerve fibers are most commonly affected, with damage to smaller sensory fibers occurring only rarely. Sensory nerve dysfunction with symptoms such as sensory ataxia, pain, and severe numbness is generally more common than motor involvement. However, motor and autonomic neuropathic symptoms may also develop. Further symptoms and characteristics of CIPN are well known in the art and are, for example, described in standard text books of medicine, such as Stedman or Pschyrembl.

Preferably, the chemotherapy-induced neuropathic pain is associated with the administration of paclitaxel and/or oxaliplatin. The term “associated with” as used herein, preferably, refers to a temporal and/or causal relationship between neuropathic pain and the administration of a chemotherapeutic agent, preferably of paclitaxel and/or oxaliplatin. A causal relationship between neuropathic pain and the administration of a chemotherapeutic agent is well known to the person skilled in the art. Preferably, the pain shall be considered to be associated with the administration of a paclitaxel and/or oxaliplatin, i.e. the pain shall be induced by said the administration of paclitaxel and/or oxaliplatin either directly or indirectly. Indication for such a causal connection is in particular a close time relationship between the administration and the pain. Typically, symptoms of chemotherapy-induced neuropathic pain may appear within 1 week, 2 weeks, 1 months, 3 months, 6 months or 1 year after administration of the chemotherapeutic agent. It is also known in the art, that the occurrence of symptoms of CIPN may be dependent on the formulation of the drug, the dosage and the administration schedule. Moreover, administration and dosage of chemotherapeutic agents, in particular paclitaxel and/or oxaliplatin, depends on various factors such as the kind and state of the cancer to be treated and the health state of the patient. Chemotherapeutic agents such as paclitaxel and/or oxaliplatin are usually administered under the supervision of a qualified physician. Preferred administration routes of chemotherapeutic agents such as paclitaxel and/or oxaliplatin include intravenous, intrathecal and intraperitoneal administration.

The term “oxidized lipid”, also referred to as “oxidized fatty acid”, as used herein relates to a lipid or fatty acid that has been oxidized by an oxygenase enzyme. Oxygenase enzymes such as COX, cyclooxygenase; LOX, lipoxygenase or CYP, Cytochrome-P450-Epoxygenase. The oxidization usually consists of the addition of a reactive group to the molecule such as an hydroxide or epoxide group. Means and methods to produce oxidized lipids/fatty acids are well known in the art. For example, oxidized lipids can be produced by reactions initiated by reactive oxygen species (ROS), such as OH. and HOO., which combines with a hydrogen atom to make water and a fatty acid radical. The resulting lipid radicals can then propagate the formation of other oxidized lipids, for example by reactions involving isomerization and chain scission. It is known in the art that oxidized lipids can have signaling functions in cells and may mediate many different biological functions, Preferably, the oxidized lipid according to the present invention is an epoxylipid.

The term “epoxylipid” or “epoxy fatty acid” as used herein relates to a fatty acid with an epoxy substituent. According to IUPAC nomenclature, an epoxy compound is a compound in which an oxygen atom is directly attached to two adjacent or non-adjacent carbon atoms of a carbon chain or ring system. The term “epoxides” is also commonly used and represents a subclass of epoxy compounds containing a saturated three-membered cyclic ether. Epoxylipids are well known in the art and include, for example, epoxyoctadecenoic acids (EpOMEs) and hydroxyoctadecadienoic acids (HODEs). Preferably, the epoxylipid according to the present invention is selected from the group consisting of 9,10-EpOME, 9,10-DiHOME, 9-HODE, 13-HODE, PGE2, PGD2, PGF2α, TXB2, LTB4, Hepoxilin A3, 5,6-EET, 5,6-DHET, 8,9-EET, 8,9-DHET, 11, 12-EET, 11, 12-DHET, 14, 14-EET, 14, 15-DHET, 12, 13-EpOME, 12. 13-DiHOME, 17, 18-EEQ, 19, 20-EDP. More preferably, the expoxylipid according to the present invention is selected from the group consisting of: 9,10-EpOME ((±)9(10)-epoxy-12Z-octadecaenoic acid), 9-HODE ((±)9-hydroxy-10(E),12(Z)-octadecadienoic acid) and 13-HODE ((±)13-hydroxy-9(Z),11(13)-octadecadienoic acid). Most preferably, the epoxylipid according to the present invention is 9,10-EpOME.

Also envisaged in accordance with the method of the present invention is that a panel of epoxylipids are determined together. More preferably, such a panel may comprise all lipids showing the same direction of change, i.e. either an increase or a decrease. A preferred panel of epoxylipids to be determined together showing an increase comprise 9,10-EpOME, 9-HODE, 9,10-DiHOME. A preferred panel of epoxylipids to be determined together showing a decrease comprise 12-S-HETE, 15-S-HETE.

The term “sample” as used herein refers to a biological sample, preferably derived from body fluids such as blood, most preferably a plasma sample. A sample, preferably a plasma sample, can be derived from a subject as specified elsewhere herein. Means and methods to obtain a plasma sample from a subject are well known in the art and include, for example, separating the plasma from erythrocytes, leucocytes and platelets contained in a blood sample. It is to be understood that a sample may be pre-treated before it is used in a method according to the present invention. Pre-treatments may include, for example, treatments required to remove excessive material or waste. Suitable techniques comprise centrifugation, extraction, fractioning, ultrafiltration, protein precipitation followed by filtration and purification and/or enrichment of compounds. Preferably, a liquid-liquid extraction technique is used to extract lipids including oxidized lipids. Moreover, other pre-treatments may be carried out in order to provide the compounds within the sample to be analyzed, i.e. oxidized lipids such as 9,10-EpOME, in a form or concentration suitable for analysis. Suitable and necessary pre-treatments depend on the means used for carrying out the method of the invention and are well known to the person skilled in the art. According to the present invention, the sample is preferably subjected to Liquid Chromatography Tandem Mass Spectrometry (LC-MS/MS), where at least one oxidized lipid, preferably an epoxylipid and most preferably 9,10-EpOME, is determined.

In a preferred embodiment of the present invention, a first sample, also named “reference sample” as described elsewhere herein, is obtained before the start of the chemotherapy, while a second sample is obtained after the start of the chemotherapy. Preferably, said reference sample is taken at least 1 months, 2 weeks, 1 week, 1 day, 12 h, 6 h, 3 h, 1 h, 30 min, 10 min, 5 min or 1 min prior to the start of the chemotherapy. Preferably, said second sample is obtained 12 h, 24 h, 48 h, 3 days, 4 days, 5 days or 1 week after the start of the chemotherapy. Most preferably, said second sample is obtained 24 h after chemotherapy.

The term “subject” as used herein relates to animals and, preferably, to mammals. More preferably, the subject is a primate and, most preferably, a human. The terms “subject” and “patient” are used interchangeably herein. The subject, preferably, is suspected to suffer from neuropathic pain, i.e. it may already show some or all of the symptoms associated with neuropathic pain. In a preferred embodiment, the subject is a patient suffering from cancer that has received a chemotherapeutic agent, preferably paclitaxel and/or oxaliplatin, and is suspected to suffer from CIPN or to have an increases risk to develop CIPN.

The term “determining” or “determining the amount” as used herein refers to determining at least one characteristic feature of at least one oxidized lipid, to be determined by the method of the present invention in the sample. Characteristic features in accordance with the present invention are features which characterize the physical and/or chemical properties including biochemical properties of an oxidized lipid Such properties include, e.g., molecular weight, viscosity, density, electrical charge, spin, optical activity, color, fluorescence, chemoluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of an oxidized lipid, by standard operations, e.g., mathematical calculations such as multiplication, division or logarithmic calculus. Most preferably, the at least one characteristic feature allows the determination and/or chemical identification of the said at least one oxidized lipid, preferably at least one expoxylipid and most preferably 9,10-EpOME, and its amount. Accordingly, the characteristic value, preferably, also comprises information relating to the abundance of the oxidized lipid from which the characteristic value is derived. For example, a characteristic value of an oxidized lipid, preferably an expoxylipid and most preferably 9,10-EpOME, may be a peak in a mass spectrum. Such a peak contains characteristic information of the oxidized lipid, preferably an expoxylipid and most preferably 9,10-EpOME, i.e. the m/z information (mass/charge ratio or quotient), as well as an intensity value being related to the abundance of the said oxidized lipid, preferably said expoxylipid and most preferably 9,10-EpOME (i.e. its amount) in the sample. Determining the amount of at least one oxidized lipid may comprise mass spectrometry or a specific chemical or biological assay. Said assay shall comprise means which allow to specifically detect the at least one oxidized lipid in the sample. Suitable assays include reporter assays, radioimmunoassays (RIA), enzyme-linked immunosorbent assay (ELISA), sandwich enzyme immune tests, electrochemiluminescence sandwich immunoassays (ECLIA), dissociation-enhanced lanthanide fluoro immuno assay (DELFIA) or solid phase immune tests. Preferably, determining the amount of at least one oxidized lipid comprises the use of mass spectrometry (MS). The term “Mass spectrometry (MS)” encompasses all techniques which allow for the determination of the molecular weight (i.e. the mass) or a mass variable corresponding to a compound, i.e. an oxidized lipid, to be determined in accordance with the present invention. Preferably, mass spectrometry as used herein relates to GC-MS, LC-MS, direct infusion mass spectrometry, FT-ICR-MS, CE-MS, HPLC-MS, quadrupole mass spectrometry, any sequentially coupled mass spectrometry such as MS-MS or MS-MS-MS, ICP-MS, Py-MS, TOF or any combined approaches using the aforementioned techniques. As an alternative or in addition to mass spectrometry techniques, the following techniques may be used for determination of at least one oxidized lipid: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). The application of the aforementioned techniques and suitable devices are well known to the person skilled in the art. Most preferably, mass spectrometry as used herein relates to LC-MS. Most preferably, determining the amount of at least one oxidized lipid as used herein comprises the use of Liquid Chromatography Tandem Mass Spectrometry (LC-MS/MS). Most preferably, determining the amount of at least one oxidized lipid as used herein comprises the use of Liquid Chromatography Tandem Mass Spectrometry (LC-MS/MS).

It will be understood that in accordance with the present invention the at least one oxidized lipid, preferably an expoxylipid and most preferably 9,10-EpOME, comprised within a sample may be, preferably, determined quantitatively or semi-quantitatively. For quantitative determination, either the absolute or precise amount of the at least one oxidized lipid will be determined or the relative amount of the at least one oxidized lipid will be determined based on the value determined for the characteristic feature(s) referred to herein above. The relative amount may be determined in a case were the precise amount of the at least one oxidized lipid can or shall not be determined. In said case, it can be determined whether the amount in which the at least one oxidized lipid is present is enlarged or diminished with respect to a second sample comprising the at least one oxidized lipid in a second amount. In a preferred embodiment said second sample comprising said the at least one oxidized lipid shall be a calculated reference or reference amount as specified elsewhere herein. Quantitatively analysing the at least one oxidized lipid, thus, also includes what is sometimes referred to as semi-quantitative analysis of an oxidized lipid.

The term “comparing” refers to determining whether the determined amount of the at least one oxidized lipid is essentially identical to a reference amount or differs therefrom. The term “reference amount” is described elsewhere herein in detail. Preferably, said at least one oxidized lipid is deemed to differ from a reference amount if the observed difference is statistically significant which can be determined by statistical techniques referred to elsewhere in this description. If the difference is not statistically significant, the amount of the at least one oxidized lipid and the reference amount are essentially identical. Based on the comparison referred to above, a subject can be assessed to suffer from. neuropathic pain, or not. Moreover, changes in the amounts may be indicated by the term “fold”-regulation” or changes in the kind of regulation may be indicated by the term “up”- or “down”-regulation, resulting in a higher or lower relative and/or absolute amount. It will be understood, that an oxidized lipid according to the present invention may be up- or downregulated compared to a reference, i.e. the amount of said oxidized lipid is increased compared to a reference amount or is decreased compared to a reference amount. Preferably, the oxidized lipids selected from the group consisting of 9,10 EpOME, 13-HODE, 9-HODE and 9,10-DiHOME will show an increased amount compared to a reference amount whereby neuropathic pain is to be diagnosed. Further preferred, the oxidized lipids selected from the group consisting of 12-S-HETE, 15-S-HETE and 6-Keto PDGF_{1α}, will show a decreased amount compared to a reference amount whereby neuropathic pain is to be diagnosed and/or predicted.

The comparison may, preferably, be assisted by automation. For example, a suitable computer program comprising algorithms for the comparison of two different data sets (e.g., data sets comprising the values of the characteristic feature(s), i.e. values relating to the amount of 9,10 EpOME), may be used. Such computer programs and algorithm are well known in the art. Notwithstanding the above, a comparison can also be carried out manually.

The term “reference amount”, also simply referred to as “reference”, relates to values of characteristic features of each of the at least one oxidized lipids which can be correlated to a medical condition, diseases status or an effect referred to herein, i.e. the presence or absence of neuropathic pain. The reference is, preferably, a threshold amount. The amount of an oxidized lipid in sample of a subject may be higher or lower than the threshold amount. A reference amount may be derived from a single subject or a group thereof. The reference may be a calculated reference, most preferably the average or median, for the relative or absolute amount of the at least one oxidized lipid of a population of individuals comprising the subject to be investigated. The absolute or relative amounts of the at least one oxidized lipid of said individuals of the population can be determined as specified else-where herein. How to calculate a suitable reference value, preferably, the average or median, is well known in the art. Preferably, the reference amount is derived from a single subject.

An altered amount for the at least one oxidized lipid found in the sample with respect to the reference is indicative for the presence of neuropathic pain. Preferably, said amount is being altered by at least 10%, by at least 20%, by at least 30%, by at least 50%. Preferably, the amount of the at least one oxidized lipid selected from the group consisting of 9,10-EpOME, 13-HODE, 9-HODE and 9,10-DiHOME, is higher than the threshold amount, while the amount of the at least one oxidized lipid selected from the group consisting of 12-S-HETE, 15-S-HETE and 6-Keto PDGF1α, is lower than the threshold amount and thus indicative for the presence of neuropathic pain. An observed difference for two amounts shall be statistically significant. A difference in the relative or absolute amount is, preferably, significant outside of the interval be-tween 45th and 55th percentile, 40th and 60th percentile, 30th and 70th percentile, 20th and 80th percentile, 10th and 90th percentile, 5th and 95th percentile, 1st and 99th percentile of the reference value. Preferably, the reference, i.e. the amount of the at least one oxidized lipid, will be stored in a suitable data storage medium such as a database and, thus, is also available for future assessments.

In accordance with the present invention, a reference amount is preferably obtained from a sample from a subject known not to suffer from neuropathic pain, i.e. an apparently healthy subject. In a preferred embodiment of the present invention, the reference amount is derived from a “reference sample” of the subject before the start of chemotherapy. It is thus to be understood that such an apparently healthy subject may be a subject suffering from cancer. A patient suffering cancer may or may not show symptoms, clinical signs or other parameters related to cancer, often depending on the stage and kind of cancer at diagnosis, and may have had other treatments before a chemotherapeutic agent such as paclitaxel and/or oxaliplatin is administered.

In a preferred embodiment of the present invention, the reference amount is derived from a subject before the start of chemotherapy, i.e. the reference amount is calculated from a “reference sample” of said subject that was taken before therapy. The amount of the oxidized lipid in the reference sample taken from the subject before the start of chemotherapy, preferably at least 1 months, 2 weeks, 1 week, 1 day, 12 h, 6 h, 3 h, 1 h, 30 min, 10 min, 5 min or 1 min prior to the start of the chemotherapy is then compared to the amount of the oxidized lipid in a sample from the subject taken after start of the chemotherapy, preferably 24 h after the start of chemotherapy. The term “predicting the risk” refers to assessing the likelihood that a disease or disorder or at least one symptom associated therewith will occur in the future, preferably said disease is neuropathic pain, more preferably chemotherapy-induced neuropathic pain (CIPN) and, most preferably, CIPN induced by paclitaxel and/or oxaliplatin. According to the present invention, the risk of developing neuropathic pain shall be predicted in a subject as defined elsewhere herein. An increased risk of developing neuropathic pain, shall, preferably lead to close monitoring and/or immediate actions or treatments for preventing neuropathic pain.

The term “prevention” or “preventing” as used herein refers to avoiding the onset of a disease or at least one symptom thereof. Preferably said disease is neuropathic pain. More preferably said disease is chemotherapy-induced neuropathic pain (CIPN). Most preferably, said CIPN is induced by paclitaxel and/or oxaliplatin. It will be understood by those skilled in the art, that “neuopathic pain” as used herein also includes pre-neuopathic pain states with no or very weak symptoms in which one or more of the symptoms required to label a person as having neuropathic pain are present and where peripheral nerve damage has not yet occurred, but the risk of developing neuropathic pain in a subject is present. Moreover, cancer patients that are about to receive or have recently received certain chemotherapeutic agent(s) typically have a risk of developing neuropathic pain, in particular chemotherapy-induced neuropathic pain (CIPN).

The term “treatment” or “treating” as used herein refers to ameliorating or curing of a disease or disorder or at least one symptom associated therewith. Preferably said disease or disorder is chronic or neuropathic pain as defined elsewhere herein. In case there is an amelioration or cure of the disease or at least a symptom associated therewith, the treatment shall be deemed to be effective. It will be understood that treating might not be effective in all subjects. However, according to the present invention it is envisaged that treatment will be effective in at least a statistically significant portion of subjects to be treated. It is well known to the skilled artisan how to determine a statistically significant portion of subjects that can be effectively treated. Whether a portion is statistically significant can be determined without further ado by the person skilled in the art using various well known statistic evaluation tools, e.g., determination of confidence intervals, p-value determination, Student's t-test, Mann-Whitney test etc. Details can be found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the finding of effective treatment will be correct for at least 60%, at least 70%, at least 80%, or at least 90% of the subjects of a given cohort or population.

Preferably, for preventing and/or treating neuropathic pain, more preferably CIPN and most preferably CIPN induced by paclitaxel and/or oxaliplatin, cytochrome P450 expoygenase (CYP)-antogonists may be administered. Preferably, for preventing CIPN, a cytochrome P450 expoygenase (CYP)-antogonists is administered after the subject has received the first dose of a chemotherapeutic agent, preferably paclitaxel and/or oxaliplatin, and before one or more symptoms of CIPN are present. Preferably, for treating CIPN, a cytochrome P450 expoygenase (CYP)-antogonists is administered after the subject has received the first dose of a chemotherapeutic agent, preferably paclitaxel and/or oxaliplatin, and one or more symptoms of CIPN have already occurred.

The definitions and explanations of the terms made above apply mutatis mutandis for the following embodiments of the present invention except specified otherwise herein below. In a preferred embodiment of the present invention, said at least one oxidized lipid is an epoxylipid.

In a further preferred embodiment of the present invention, said at least one expoxylipid is selected from the group consisting of: 9,10-EpOME ((±)9(10)-epoxy-12Z-octadecaenoic acid), 9-HODE ((±)9-hydroxy-10(E),12(Z)-octadecadienoic acid) and 13-HODE ((±)13-hydroxy-9(Z),11(E)-octadecadienoic acid). Most preferably, said at least one expoxylipid is 9,10-EpOME ((±)9(10)-epoxy-12Z-octadecaenoic acid).

In yet a preferred embodiment of the present invention, said neuropathic pain is chemotherapy-induced neuropathic pain (CIPN).

In yet a preferred embodiment of the present invention, said. CIPN is induced by paclitaxel and/or oxaliplatin.

In yet a preferred embodiment of the present invention, said amount of the at least one oxidized lipid is determined 24 h after the start of chemotherapy.

In yet a preferred embodiment of the present invention, said reference amount corresponds to the amount of said at least one oxidized lipid before the start of chemotherapy.

Moreover, the present invention relates to a method for predicting whether a subject is at risk of developing neuropathic pain comprising the steps of

(a) determining in a plasma sample of the subject the amount of at least one oxidized lipid;

(b) comparing the amount of the said at least one oxidized lipid to a reference amount, whereby it is predicted whether a subject is at risk of developing neuropathic pain.

In a preferred embodiment of the present invention, said at least one oxidized lipid is an epoxylipid, preferably said epoxylipid is selected from the group consisting of: 9,10-EpOME ((±)9(10)-epoxy-12Z-octadecaenoic acid), 9-HODE ((±)9-hydroxy-10(E),12(Z)-octadecadienoic acid) and 13-HODE ((±)13-hydroxy-9(Z),11(E)-octadecadienoic acid). Most preferably, said at least one expoxylipid is 9,10-EpOME ((±)9(10)-epoxy-12Z-octadecaenoic acid).

In a further preferred embodiment of the present invention, said neuropathic pain is chemotherapy-induced neuropathic pain (CIPN), preferably induced by paclitaxel and/or oxaliplatin.

In yet a preferred embodiment of the present invention, said amount of the at least one oxidized lipid is determined 24 h after the start of chemotherapy.

The present invention also encompasses a device for carrying out a method according to any one of claims 1 to 12, comprising:

a) an analyzing unit comprising at least one detector for at least one oxidized lipid as predictive and/or diagnostic biomarker, wherein said analyzing unit is adapted for determining the amount of at least one oxidized lipid as predictive and/or diagnostic biomarker by the at least one detector, and, operatively linked thereto;

b) an evaluation unit comprising a computer comprising tangibly embedded a computer program code for carrying out a comparison of the determined amount of the at least one oxidized lipid as predictive and/or diagnostic biomarker, preferably 9,10-EpOME, with a reference and a data base comprising said reference for said at least one oxidized lipid as predictive and/or diagnostic biomarker, whereby it is predicted and/or diagnosed whether a subject suffers from neuropathic pain.

The term “device” as used herein relates to an apparatus or system that shall comprise at least the aforementioned means. Moreover, the device, preferably, further comprises means for comparison and evaluation of the detected characteristic feature(s) of the at least one oxidized lipid and, also preferably, the determined amount. The means of the device are, preferably, operatively linked to each other. How to link the means in an operating manner will depend on the type of means included into the device. For example, where means for automatically qualitatively or quantitatively determining the amount of at least one oxidized lipid as predictive and/or diagnostic biomarker are applied, the data obtained by said automatically operating means can be processed by, e.g., a computer program in order to facilitate the assessment. Preferably, the means are comprised by a single device in such a case. Said device may accordingly include an analysing unit comprising at least one detector for at least one oxidized lipid and an evaluation unit comprising a computer for processing the resulting data for the assessment. Preferred devices are those which can be applied without the particular knowledge of a specialized clinician, e.g., electronic devices which merely require loading with a plasma sample.

Alternatively, the methods for diagnosing and/or predicting neuropathic pain comprising the determination of the at least one oxidized lipid can be implemented into a system comprising several devices which are, preferably, operatively linked to each other. Specifically, the means must be linked in a manner as to allow carrying out the methods of the present invention as described in detail above. The term “operatively linked” as used herein thus, preferably, means functionally linked. Depending on the means to be used for the system of the present invention, said means may be functionally linked by connecting each mean with the other by means which allow data transport in between said means, e.g., glass fiber cables, and other cables for high throughput data transport. Nevertheless, wireless data transfer between the means is also envisaged by the present invention, e.g., via LAN (Wireless LAN, W-LAN). A preferred system comprises means for determining oxidized lipids such as chromatographic devices, and mass spectrometry devices as described elsewhere herein. Further comprised shall be means for comparing and/or analyzing the results obtained from the means for determination of the at least one oxidized lipid. The means for comparing and/or analyzing the results may comprise at least one database and an implemented computer program for comparison of the results.

The present invention also relates to a method for determining whether a neuropathic pain therapy is successful comprising the steps of:

a) determining at least one oxidized lipid in a first and a second sample of the subject wherein said first sample has been taken prior to or at the onset of the neuropathic pain therapy and said second sample has been taken after the onset of the said therapy; and

b) comparing the amount of the said at least one oxidized lipid in the first sample to the amount in the second sample, whereby a change in the amount determined in the second sample in comparison to the first sample is indicative for neuropathic pain therapy being successful.

In a preferred embodiment of the present invention, said neuropathic pain therapy comprises administration of a cytochrome P450 expoygenase (CYP)-antagonist.

Moreover, the present invention pertains to an oxidized lipid for use in a method for diagnosing neuropathic pain and/or in a method for predicting whether a subject is at risk of developing neuropathic pain and/or in method for determining whether a neuropathic pain therapy is successful according to the present invention.

Further embodiments of the present invention concern:

An oxidized lipid for use in the diagnosis, prevention or treatment of neuropathic pain in a subject. In some embodiments of the invention, the lipid is an epoxylipid. In some other embodiments of the invention, the lipid can be selected from the group consisting: 9,10-EpOME, 9,10-DiHOME, 9-HODE, 13-HODE, PGE₂, PGD₂, PGF_{2α}, TXB₂, LTB₄, Hepoxilin A₃, 5,6-EET, 5,6-DHET, 8,9-EET, 8,9-DHET, 11, 12-EET, 11, 12-DHET, 14, 14-EET, 14, 15-DHET, 12, 13-EpOME, 12, 13-DiHOME, 17, 18-EEQ, 19, 20-EDP.

In a further embodiment, the expoxylipid is, preferably, selected from the group consisting of: 9,10-EpOME ((±)9(10)-epoxy-12Z-octadecaenoic acid), 9-HODE ((±)9-hydroxy-10(E),12(Z)-octadecadienoic acid) and 13-HODE ((±)13-hydroxy-9(Z),11(E)-octadecadienoic acid), most preferably is 9,10-EpOME ((±)9(10)-epoxy-12Z-octadecaenoic acid).

Further preferred embodiments of the present invention include:

The lipid for use in the diagnosis, prevention or treatment of neuropathic pain in a subject, wherein said neuropathic pain is selected from the group consisting of post-herpetic neuralgia, trigeminal neuralgia, focal peripheral nerve injury, and anesthesia dolorosa, central pain due to stroke or mass lesion, spinal cord injury, or multiple sclerosis, and peripheral neuropathy due to diabetes, HIV, or chemotherapy.

In context of the herein described invention said pain to be treated is preferably neuropathic pain (including pain associated with diabetic neuropathy, postherpetic neuralgia, HIV/AIDS induced neuropathic pain, traumatic injury, complex regional pain syndrome, trigeminal neuralgia, erythromelalgia and phantom pain), pain produced by mixed nociceptive and/or neuropathic mixed etiologies (e.g., cancer), osteoarthritis, fibromyalgia, lower back pain, inflammatory hyperalgesia, vulvar vestibulitis or vulvodynia, sinus polyps interstitial cystitis, neurogenic or overactive bladder, prostatic hyperplasia, rhinitis, surgery, trauma, rectal hypersensitivity, burning mouth syndrome, oral mucositis, herpes (or other viral infections), prostatic hypertrophy, dermatitis, pruritis, itch, tinnitus, psoriasis, warts, cancers, headaches, and wrinkles, central pain due to stroke or mass lesion, spinal cord injury, or multiple sclerosis. However, most preferred embodiments pertain to chemotherapy-induced peripheral neuropathic pain (CIPNP).

The present invention also provides the use of the oxidized lipid as above stated as a biomarker in a method for the prediction of onset, intensity or duration of neuropathic pain in a subject.

In another embodiment of the present invention, the concentration of the oxidized lipid is measured after 24 hours after the start of chemotherapy.

In a further embodiment of the invention, the concentration of the oxidized lipid is measured from plasma. In another embodiment, the concentration is measured using LC-MS/MS.

According to the present invention, the treatment of the pain in a subject starts when the concentration of the epoxylipid and/or oxidized lipid is at least 20%, preferably 30%, more preferably 40% higher than the normal value.

Further provided by the present invention is a method for the prediction of onset, intensity or duration of neuropathic pain in a subject, comprising:

a) separating plasma from blood sample;

b) measuring the concentration of an oxidized lipid in the plasma;

c) determining the ratio of the elevation of the oxidized lipid.

In a preferred embodiment of the invention, the oxidized lipid is an epoxylipid. The expoxylipid is preferably selected from the from the group consisting of: 9,10-EpOME ((±)9(10)-epoxy-12Z-octadecaenoic acid), 9-HODE ((±)9-hydroxy-10(E),12(Z)-octadecadienoic acid) and 13-HODE ((t)13-hydroxy-9(Z),11(E)-octadecadienoic acid), most preferably is 9,10-EpOME ((±)9(10)-epoxy-12Z-octadecaenoic acid).

As a preferred embodiment, the concentration of the oxidized lipid is measured as early as 24 hours after the beginning of a chemo-therapy.

The present invention further provides a therapeutic method for the treatment of neuropathic pain in a subject, characterized in that the therapy starts before the first symptoms arise in patient. A preferred therapeutic method for the treatment of neuropathic pain is to use a cytochrome P450 expoygenase (CYP)-antagonist for the treatment of neuropathic pain. In a preferred embodiment, the therapy starts after the elevation of an oxidized lipid has been detected according to the method of this invention.

A preferred therapeutic method of the present invention is a cytochrome P450 epoxygenase (CYP)-antagonist for use in the prevention or treatment of pain in a subject. In some embodiments of the invention the CYP-antagonist is selected from the group consisting of a CYP1A-, CYP2B-, CYP2C-, CYP2E-, and preferably a CYP2J-antagonist. Most preferably the CYP-antagonist is an antagonist of a mammalian homologue of CYP2J2 (CYP2J2-antagonist), preferably hu-man CYP2J2, such as telmisartan, aripiprazole or most preferably terfenadine.

A further preferred therapeutic method of the invention is the use of any CYP2J2 antagonist, preferably a selective CYP2J2 antagonist. The term “selective CYP2J2 antagonist” pertains to antagonists of CYP2J2 that selectively inhibit activity, function or expression of CYP2J2 but not of other related enzymes such as for example CYP3A molecules. To identify whether a candidate antagonist is a CYP2J2 antagonist, a luminogenic cytochrome P450 glow assay can be employed. CYP proteins catalyse the formation of arachidonic acid metabolites. Luminogenic CYP assays use prosubstrates for the light-generating reaction of luciferase. CYPs convert the prosubstrates to luciferin or a luciferin ester, which produces light in a second reaction with a luciferase reaction mix called Luciferin Detection Reagent (LDR). The amount of light produced in the second reaction is proportional to CYP activity.

Another pain therapy comprising the inhibition of the activity of in particular CYP2J2 which produces the metabolic compound 9,10-EpOME—according to the invention, a sensitizer of ion channel-mediated pain perception. Surprisingly, the inhibition of CYP2J2 in accordance with the invention proved to be effective in-viva to alleviate neuropathic pain induced by paclitaxel in a mouse model, indicating the use of CYP2J2 antagonists as analgesic against neuropathic pain, in particular CIPNP.

One further embodiment of the invention relates to the abovementioned prevention or treatment of pain, which comprises the administration of said CYP antagonist of the invention to a subject suffering from said pain, and wherein said subject received, receives or will receive chemotherapy. Therefore, the subject is in preferred embodiments a subject suffering from, or diagnosed with, a cancer disease.

Chemotherapy in context of the invention preferably involves the administration of a chemotherapeutic agent to a subject in need of such a treatment selected from pyrimidinone-based anti-neoplastic agents such as cytarabine, 5-flurouracil or platin agents, such as cisplatin, or taxanes, such as paclitaxel, docetaxel or cabazitaxel, or derivatives thereof. Such chemotherapeutic agents are known to induce neuropathic pain, in particular this is known for taxanes, which are therefore preferred in context of the invention. Most preferred is paclitaxel.

In animal models of neuropathic pain [4, 5], we use LC-MS/MS to perform lipid profiling and surprisingly observed increased concentrations of specific epoxylipids and oxidized lipids from the arachidonic and linoleic acid pathway, Particularly, the concentrations of 9,10-EpOME ((±)9(10)-epoxy-12Z-octadecaenoic acid), 9-HODE ((±)9-hydroxy-10(E),12(Z)-octadecadienoic acid) and 13-HODE ((±)13 -hydroxy-9(Z), 11(E)-octadecadienoic acid) (FIG. 1-3) are found to be elevated in various pain relevant tissues and in the plasma following paclitaxel- or oxaliplatin-treatment which leads to peripheral neuropathic pain. These increased concentrations can already be measured as early as 24 hours after paclitaxel or oxaliplatin-injection in mice, even before the animals develop pain (FIG. 1). Usually, it takes around seven days until the peripheral neuropathy is developed in these models (FIG. 1, right) [4-6]. Moreover, the concentrations of the arachidonic acid metabolites 12-HETE ((±)12-hydroxy-5Z,8Z,10E,14Z-eicosatetraenoic acid), 15-HETE ((±)15 -hydroxy-5Z,8Z,11Z,13E-eicosatetraenoic acid) und 6-keto-Prostaglandin-F_{1α} are found to be decreased in pain relevant tissues (FIG. 2). Taken together, these results imply a mass spectrometric based quantification of the ratio of 9,10-EpOME and other oxidized lipids in plasma as promising biomarkers for predicting increased risks for the development of neuropathic pain. If this is successful in patients, a physician could for example treat patients with increased 9,10-EpOME or 9-HODE concentrations in the plasma preventatively to reduce or even prevent the occurrence of neuropathic pain.

9,10-EpOME and the other oxidized lipids can be measured easily from plasma (FIG. 4). Only a small volume of about 100 μl is needed for this analysis. Cancer patients that suffer from adverse events of chemotherapy or diabetes patients have to undergo blood analysis regularly, so that a small part of blood could be used for lipid anaylsis without additional stress or distraction. Measuring lipids with LC-MS/MS is a standard method (described below) that has already been established in many labs, so that the analysis of oxidized lipids from plasma can be performed rapidly, usually within a day after obtaining plasma from patients. Other methods to measure these biomarkers or their ratios, such as enzyme immune assays as a commercially available kit can also be used for the detection of these lipids.

TABLE 1
Formal chemical classification of putative
lipid biomarkers

| Abbre- | | | |
|---|---|---|---|
| viated | | | Cas- |
| name | Formal name | InChIKey | number |
| 9,10- | (±)9(10)-epoxy-12Z- | FBUKMFOXMZRGRB- | 6814-52-4 |
| EpOME | octadecaenoic acid | XKJZPFPASA-N | |
| 9,10- | (±)9(10)-dihydroxy-12Z- | XEBKSQSGNGRGDW- | 263399-34-4 |
| DiHOME | octadecenoic acid | CJWPDFJNSA-N | |
| 9-HODE | (±)-9-hydroxy-10E,12Z- | NPDSHTNEKLQQIJ- | 98524-19-7 |
| | octadecadienoic acid | ZJHFMPGASA-N | |
| 13-HODE | (±)-13-hydroxy-9Z,11E- | HNICUWMFWZBIFP- | 73804-64-5 |
| | octadecadienoic acid | BSZOFBHHSA-N | |
| 12-HETE | (±)12-hydroxy-5Z,8Z,10E, | ZNHVWPKMFKADKW- | 71030-37-0 |
| | 14Z-eicosatetraenoic acid | VXBMJZGYSA-N | |
| 15-HETE | (±)15-hydroxy-5Z,8Z,11Z, | JSFATNQSLKRBCI- | 73836-87-0 |
| | 13E-eicosatetraenoic acid | USWFWKISSA-N | |
| 6-keto- | 6-oxo-9α,11α,15S-trihydroxy- | KFGOFTHODYBSGM- | 58962-34-8 |
| PGF_{1α} | prost-13E-en-1-oic acid | ZUNNJUQCSA-N | |

TABLE 2
Group of oxidized lipids measured by LC-MS/MS

| | | | |
|---|---|---|---|
| Abbreviated | | | Cas- |
| name | Formal name | InChIKey | number |

2a: COX-metabolites

| | | | |
|---|---|---|---|
| PGE2, | 9-oxo-11.alpha.,15S- | XEYBRNLFEZDVAW- | 363-24-6 |
| Prostaglandin | dihydroxy-prosta-5Z,13E- | ARSRFYASSA-N | |
| E₂ | dien-1-oic acid | | |
| PGD2, | 9α,15S-dihydroxy-11-oxo- | BHMBVRSPMRCCGG- | 41598-07-6 |
| Prostaglandin | prosta-5Z,13E-dien-1- | OUTUXVNYSA-N | |
| D₂ | oic acid | | |
| PGF2, | 9α,11α,15S-trihydroxy-prosta- | PXGPLTODNUVGFL- | 551-11-1 |
| Prostaglandin | 5Z,13E-dien-1-oic acid | YNNPMVKQSA-N | |
| F_{2α} | | | |
| TXB2, | 9α,11,15S-trihydroxythromba- | XNRNNGPBEPRNAR- | 54397-85-2 |
| Thromboxane B₂ | 5Z,13E-dien-1-oic acid | JQBLCGNGSA-N | |
| 6-keto-PGF_{1α} | see above (Table 1) | see above | see above |
| | | (Table 1) | (Table 1) |

2b: LOX-metabolites

| | | | |
|---|---|---|---|
| LTB₄, | 5S,12R-dihydroxy- | VNYSSYRCGWBHLG- | 71160-24-2 |
| Leukotriene B₄ | 6Z,8E,10E,14Z- | AMOLWHMGSA-N | |
| | eicosatetraenoic acid | | |
| 12-HETE | see above (Table 1) | see above | see above |
| | | (Table 1) | (Table 1) |
| 15-HETE | see above (Table 1) | see above | see above |
| | | (Table 1) | (Table 1) |
| 20-HETE | 20-hydroxy-5Z,8Z,11Z,14Z- | NNDIXBJHNLFJJP- | 9551-86-3 |
| | eicosatetraenoic acid | DTLRTWKJSA-N | |
| Hepoxilin A3 | (5Z,9E,14Z)-(11S,12S)-11,12- | SGTUOBURCVMACZ- | 85589-24-8 |
| | Epoxy-8-hydroxyicosa-5,9,14- | CIODQOFUSA-N | |
| | trienoic acid | | |

2c: CYP-metabolites

| | | | |
|---|---|---|---|
| 5,6-EET | (±)5(6)-epoxy-8Z,11Z,14Z- | VBQNSZQZRAGRIX- | 87173-80-6 |
| | eicosatrienoic acid | GSKBNKFLSA-N | |
| 5,6-DHET | (±)5,6-dihydroxy-8Z,11Z,14Z- | GFNYAPAJUNPMGH- | 213382-49-1 |
| | eicosatrienoic acid | GSKBNKFLSA-N | |
| 8,9-EET | (±)8(9)-epoxy-5Z,11Z,14Z- | DBWQSCSXHFNTMO- | 81246-85-7 |
| | eicosatrienoic acid) | ZZMPYBMWSA-N) | |
| 8,9-DHET | (±)8,9-dihydroxy-5Z,11Z,14Z- | DCJBINATHQHPKO- | 192461-96-4 |
| | eicosatrienoic acid | ZZMPYBMWSA-N | |
| 11,12-EET | (±)11(12)-epoxy-5Z,8Z,14Z- | DXOYQVHGIODESM- | 123931-40-8 |
| | eicosatrienoic | LZXKBWHHSA-N | |
| | acid | | |
| 11,12-DHET | (±)11,12-dihydroxy- | LRPPQRCHCPFBPE- | 192461-95-3 |
| | 5Z,8Z,14Z-eicosatrienoic | LZXKBWHHSA-N | |
| | acid | | |
| 14,15-EET | (±)14(15)-epoxy-5Z,8Z,11Z- | JBSCUHKPLGKXKH- | 81276-03-1 |
| | eicosatrienoic | KZTFMOQPSA-N | |
| | acid | | |
| 14,15-DHET | (±)14,15-dihydroxy- | SYAWGTIVOGUZMM- | 77667-09-5 |
| | 5Z,8Z,11Z-eicosatrienoic | KZTFMOQPSA-N | |
| | acid | | |
| 9,10-EpOME | see above (Table 1) | see above | see above |
| | | (Table 1) | (Table 1) |
| 9,10-DiHOME | see above (Table 1) | see above | see above |
| | | (Table 1) | (Table 1) |
| 12,13-EpOME | (±)12(13)epoxy-9Z- | CCPPLLJZDQAOHD- | 503-07-1 |
| | octadecenoic acid | GJGKEFFFSA-N | |
| 12,13-DiHOME | 12,13-dihydroxy-9Z- | CQSLTKIXAJTQGA- | 263399-35-5 |
| | octadecenoic acid | GJGKEFFFSA-N | |
| 17,18-EEQ | (±)17(18)-epoxy- | GPQVVJQEBXAKBJ- | ?? |
| | 5Z,8Z,11Z,14Z- | YQLHGUCYSA-N | |
| | eicosatetraenoic acid | | |
| 19,20-EDP | (±)19(20)-epoxy- | OSXOPUBJJDUAOJ- | ?? |
| | 4Z,7Z,10Z,13Z,16Z- | MWEXLPNRSA-N | |
| | docosapentaenoic | | |
| | acid | | |

In the following, certain terminology used in this document is further explained:

Oxidized lipid: A lipid (fatty acid) that has been oxidized by an oxygenase enzyme (COX, cyclooxygenase; LOX, lipoxygenase or CYP, Cytochrome-P₄₅₀-Epoxygenase). These oxidized lipids have signaling functions in cells and mediate many different biological functions. The oxidization usually consists of the addition of a reactive group to the molecule (usually hydroxide or epoxide group→epoxylipid).

Peripheral neuropathic pain: A disturbance of function or pathological change in a sensory nerve causing pain. This is mediated by a lesion or disease of the peripheral somatosensory nervous system and usually appears as pain in the extremities (feet or hands) caused by light mechanical stimulations (such as touch) or cold temperatures. Peripheral neuropathic pain, may as well appear without stimulations (spontaneous pain).

Chemotherapy-induced neuropathic pain: Neuropathic pain that is a side effect (adverse event) of a cancer therapy and is caused by the toxicity of cancer therapeutics.

Cytostatics: pharmacological substances for the treatment of cancer, such as paclitaxel, or oxaliplatin.

Amitriptyline, gabapentin and duloxetine: Drugs that are approved for the treatment of neuropathic pain.

LC-MS/MS: liquid chromatography-tandem mass spectrometry, a coupled analytical method for the specific determination and quantification of low molecular weight analytes in biological samples.

InChIKey: International Chemical identifier for chemical substances that has been employed by the TUPAC (international Union of Pure and Applied Chemistry) for the specific identification of chemical substances.

All references cited throughout this specification are herewith incorporated by reference with respect to their entire disclosure content or the disclosure content referred to in a specific context.

### FIGURES

FIG. 1: (A) Concentrations of 9,10-EpOME in nervous tissue (sciatic nerve, lumbar dorsal root ganglia (DRGs) and dorsal horn of the spinal cord 24 h after i.p.-injection of paclitaxel (6 mg/kg) or oxaliplatin (3mg/kg). Data are shown as mean±SEM from five mice per group; one-way ANOVA, *p<0.05, ***p<0.001. (B) time-course of mechanical allodynia after paclitaxel-injection in mice.

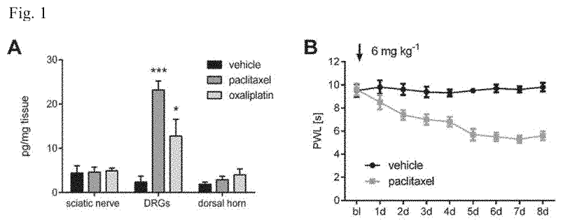

FIG. 2: (A) Concentrations of 9,10-EpOME in nervous tissue (sciatic nerve, lumbar dorsal root ganglia (DRGs) and dorsal horn of the spinal cord 8 d after multiple i.p.-injection of paclitaxel (4×2 mg/kg, injection every other day). (B) Concentrations of 6-keto-PGF_{1α} in nervous tissue (sciatic nerve, lumbar dorsal root ganglia and dorsal horn of the spinal cord 8 d after multiple i.p.-injection of paclitaxel (4×2 mg/kg, injection every other day). Concentrations of 12S-(C) and 15S-HETE (D) 8 d after i.p.-injection of paclitaxel (6 mg/kg) in nervous tissue. Data are shown as mean±SEM from five mice per group; one-way ANOVA, *p<0.05, **p<0.01, n.d: not determined.

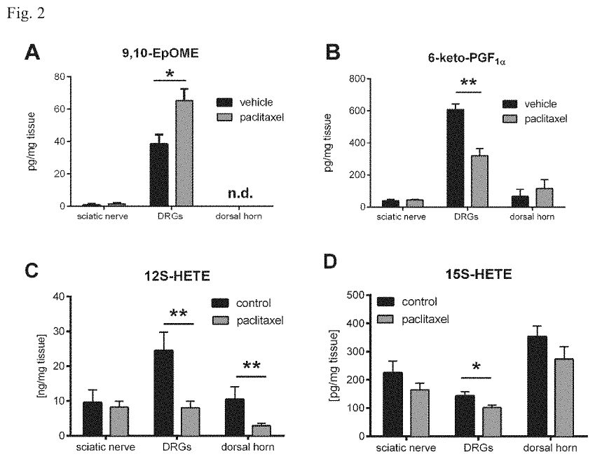

FIG. 3: Concentrations of 13-HODE (A)and 9-HODE (B) in nervous tissue (sciatic nerve, lumbar dorsal root ganglia (DRGs) and dorsal horn of the spinal cord 10 d after i.p.-injection of oxaliplatin (3 mg/kg, injection every other day). Data are shown as mean±SEM from five mice per group; one-way ANOVA, *p<0.05.

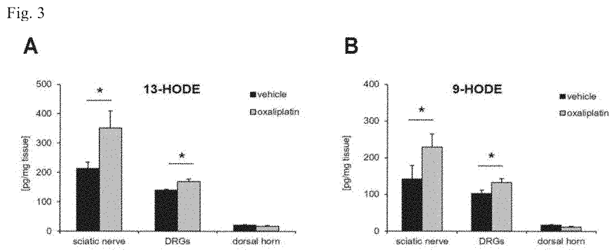

FIG. 4: Plasma-concentrations of 9,10-EpoME and its Metabolite 9,10-DiHOME 8d after paclitaxel-treatment (6 mg/kg) can be reduced by administration of telmisartan (10 mg/kg, 2h). Data are shown as mean±SEM from five mice per group; one-way ANOVA, *p<0.05.

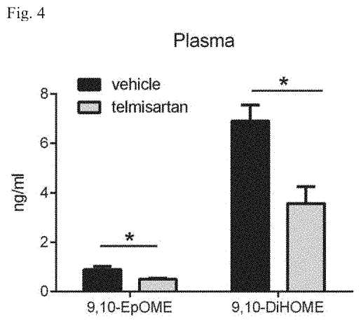

The invention will now be described by way of Examples. However, the Examples shall not be construed, whatsoever, as limiting the scope of the invention.

### EXAMPLES

### Example 1: Models of Chemotherapy-Induced Peripheral Neuropathic Pain

All animal experiments were performed according to the recommendations in the Guide for the Care and Use of Laboratory Animals of the National Institutes of Health and approved by the local Ethics Committees for Animal Research (Darmstadt) with the permit number F95/42. For all behavioral experiments, only 6-12 weeks old male C57BL/6N mice were used and purchased from commercial breeding companies (Charles River, Sulzfeld, Germany, Janvier, Le Geneset-Saint-Isle, FR). To compare mechanical thresholds, age and sex matched littermates were used as control.

Paclitaxel was dissolved in Cremophor EL/Ethanol 1:1 and diluted in saline. The dose for intraperitoneal injection was set to 6 mg/kg as described previously [4]. Oxaliplatin was dissolved in saline. The dose for intraperitoneal injection was set to 3 mg/kg as described previously [5].

### Example 2: Measurement of Oxidized Lipids From Plasma Using LC-MS/MS

### Standards and Internal Standards

For the measurement of epoxy lipids and HETEs stock solutions of the analytes 9,10-EpOME, 9-HODE, 13-HODE, 12-HETE and 15-HETE and internal standards 9,10-EpOME-d4, 9-HODE-d4, 13-HODE-d4, 12-HETE-d4 and 15-HETE-d4 are generated with concentrations of 2500 ng/ml in ethanol. Working standards were obtained by further dilution with a concentration range of 0.1-250 ng/ml for all analytes.

For prostanoids, stock solutions with 50,000 ng/ml of all analytes (6-keto-PGF_{1α}) and internal standards (6-keto-PGF_{1α}-d4) were prepared in methanol. Working standards were obtained by further dilution with a concentration range of 0.1-1,250 ng/ml

### Lipid-Extraction From Plasma

Lipids are extracted twice with 600 μl of ethyl acetate using liquid-liquid extraction. The combined organic phases were removed at a temperature of 45° C. under a gentle stream of nitrogen. The residues were reconstituted with 50 μl of methanol/water/butylated hydroxytoluene (BHT) (50:50:10⁻³, v/v/v) (EpOMEs, HODEs and HETEs), or 50 μl of acetonitrile/water/formic acid (20:80:0.0025, v/v/v) (6-keto-PGF_{1α}) and then centrifuged for 2 min at 10,000 g, and transferred to glass vials waiting for analysis.

### Instrumentation For Lipid Measurement

The LC-MS/MS system consists of a QTrap 5500 (AB Sciex, Darmstadt, Germany) equipped with a Turbo-V source operating in negative electrospray ionization mode, an Agilent 1200 binary HPLC pump and degasser (Agilent, Waldbronn, Germany), and an HTC Pal autosampler (CTC analytics, Zwingen, Switzerland). High-purity nitrogen for the mass spectrometer was produced by a NGM 22-LC-MS nitrogen generator (cmc Instruments, Eschborn, Germany).

For the chromatographic separation of EpOMEs, HODEs and HETEs, a Gemini NX C18 column and precolumn were used (150×2 mm inner diameter, 5 μm particle size, and 110 Å pore size; Phenomenex, Aschaffenburg, Germany). For the chromatographic separation of prostanoids, a Synergi 4 u Hydro-RP column (150×2 mm inner diameter, 4 μm, Phenomenex, Aschaffenburg, Germany) and a precolumn of same material were used.

### LC-Gradient and Data Analysis

For measurements of EpOMEs, HODEs und HETEs a linear gradient was used at a flow rate of 0.5 ml/min with a total run time of 17.5 min. Mobile phase A consist of water: ammonia (100:0.05, v/v), and mobile phase B of acetonitrile ammonia (100:0.05, v/v). The gradient changed from 85% A to 10% within 12 min. These conditions were held for 1 min. Then, the mobile phase shifted back to 85% A within 0.5 min and it was maintained for 4 min to re-equilibrate the column.

For the chromatographic separation of prostanoids, a Synergi 4 u Hydro-RP column (150×2 mm inner diameter, 4 μm, Phenomenex, Aschaffenburg, Germany) and a precolumn of same material were used. Chromatographic separation was carried out in gradient elution mode at a flow rate of 0.3 ml/min, Total run time was 16 min Mobile phase A consisted of water/formic acid (100:0.0025, v/v), and mobile phase B of acetonitrile/formic acid (100:0.0025, v/v). The linear gradient started with 90% A for 1 min and then changed to 60% A within 1 min, It was held for 1 min at 60% in phase A. Within 1 min, the mobile phase shifted to 50% in phase A and was held for 2 min. Within 2 min, the mobile phase shifted to 10% A and was held for 1 min. Composition of the gradient shifted back to 90% A in one min and it was maintained for 6 min to re-equilibrate the column.

A volume of 20 μl (EpOMEs, HODEs und HETEs) or 45 μl (prostanoids) of the extracted samples was injected into the LC-MS/MS system. Quantification was performed with Analyst software version 1.6 (Applied Biosystems) using the internal standard method (isotope-dilution mass spectrometry). Ratios of analyte peak area and internal standard area. (y-axis) were plotted against concentration (x-axis), and calibration curves were calculated by least-squares regression with 1/square concentration weighting.

### CITED REFERENCES

1. Finnerup N B, Attal N, Haroutounian S, McNicol E, Baron R, Dworkin R. H, Gilron I, Haanpaa M, Hansson P, Jensen T S et al: Pharmacotherapy for neuropathic pain in adults: a systematic review and meta-analysis. Lancet Neurol 2015, 14(2):162-173.

2. Dworkin R H, O'Connor A B, Audette J, Baron R, Gourlay G K, Haanpaa M L, Kent J L, Krane E J, Lebel A A, Levy R M et al: Recommendations for the pharmacological management of neuropathic pain: an overview and literature update. Mayo Gin Proc 2010, 85(3 Suppl):53-14.

3. Borsook D, Becerra L, Hargreaves Biomarkers for chronic pain and analgesia. Part 1: the need, reality, challenges, and solutions. Discov Med 2011, 11(58):197-207.

4. Alessandri-Haber N, Dina O A, Joseph E K, Reichling D B, Levine J D: Interaction of transient receptor potential vanilloid 4, integrin, and SRC tyrosine kinase in mechanical hyperalgesia. J Neurosci 2008, 28(5):1046-1057.

5. Nassini R, Gees M, Harrison S, De Siena G, Materazzi S, Moretto N, Failli P, Preti D,

Marchetti N, Cavazzini A et al: Oxaliplatin elicits mechanical and cold allodynia in rodents via TRPA1. receptor stimulation. Pain 2011, 152(7):1621-1631.

6. Materazzi S, Fusi C, Benemei S, Pedretti P, Patacchini R, Nilius B, Prenen J, Creminon C. Geppetti P. Nassini R: TRPA1 and TRPV4 mediate paclitaxel-induced peripheral neuropathy in mice via a glutathione-sensitive mechanism. Pflugers Arch 2012, 463(4):561-569.

7. Bennett D L, Woods C G: Painful and painless channelopathies. Lancet Neurol 2014.

8. Kremeyer B, Lopera F, Cox J J, Momin A, Rugiero F, Marsh S, Woods C G, Jones N G, Paterson K J, Fricker F R et al: A gain-of-function mutation in TRPA1 causes familial episodic pain syndrome. Neuron 2010, 66(5):671-680.

9. Inceoglu, Bora, et al. “Acute augmentation of epoxygenated fatty acid levels rapidly reduces pain-related behavior in a rat model of type I diabetes.” Proceedings of the National Academy of Sciences 109.28 (2012): 11390-11395.

10. WO2010/062900.

11. WO2009/062073.

12. Komori, Shunji, et al. “A novel role of prostaglandin E2 in neuropathic pain.” Glia 59.2 (2011): 208-218.

13. Ramsden, Christopher E., et al. “Targeted alteration of dietary n-3 and n-6 fatty acids for the treatment of chronic headaches: a randomized trial.” PAIN® 154.11 (2013): 2441-2451.

1. A method for diagnosing neuropathic pain in a subject comprising:
(a) determining in a plasma sample of a subject suspected to suffer from neuropathic pain the amount of at least one oxidized lipid;
(b) comparing the said amount of the at least one oxidized lipid with a reference amount, whereby neuropathic pain is to be diagnosed.

2. The method of claim 1, wherein said at least one oxidized lipid is an epoxylipid.

3. The method of claim 1, wherein said at least one expoxylipid is selected from the group consisting of: 9,10-EpOME ((±)9(10)-epoxy-12Z-octadecaenoic acid), 9-HODE ((±)9-hydroxy-10(E),12(Z)-octadecadienoic acid) and 13-HODE ((±)13-hydroxy-9(Z),11(E)-octadecadienoic acid).

4. The method of claim 1, wherein said neuropathic pain is chemotherapy-induced neuropathic pain (CIPN).

5. The method of claim 1, wherein said CIPN is induced by paclitaxel and/or oxaliplatin.

6. The method of claim 4, wherein the amount of the at least one oxidized lipid is determined 24 h after the start of chemotherapy.

7. The method of claim 4, wherein said reference amount corresponds to the amount of said at least one oxidized lipid before the start of chemotherapy.

8. A method for predicting whether a subject is at risk of developing neuropathic pain comprising:
(a) determining in a plasma sample of the subject the amount of at least one oxidized lipid;
(b) comparing the amount of the said at least one oxidized lipid to a reference amount, whereby it is predicted whether a subject is at risk of developing neuropathic pain.

9. The method of claim 8, wherein said at least one oxidized lipid is an epoxylipid.

10. The method of claim 8, wherein said neuropathic pain is chemotherapy-induced neuropathic pain (CIPN).

11. The method of claim 8, wherein the amount of the at least one oxidized lipid is determined 24 h after the start of chemotherapy.

12. A device for carrying out a method according to claim 1, comprising:
a) an analysing unit comprising at least one detector for at least one oxidized lipid as predictive and/or diagnostic biomarker, wherein said analyzing unit is adapted for determining the amount of at least one oxidized lipid as predictive and/or diagnostic biomarker by the at least one detector; and, operatively linked thereto
b) an evaluation unit comprising a computer comprising tangibly embedded a computer program code for carrying out a comparison of the determined amount of the at least one oxidized lipid as predictive and/or diagnostic biomarker, with a reference and a data base comprising said reference for said at least one oxidized lipid as predictive and/or diagnostic biomarker, whereby it is predicted and/or diagnosed whether a subject suffers from neuropathic pain.

13. A method for determining whether a neuropathic pain therapy is successful, the method comprising:
a) determining at least one oxidized lipid in a first and a second sample of the subject wherein said first sample has been taken prior to or at the onset of the neuropathic pain therapy and said second sample has been taken after the onset of the said therapy; and
b) comparing the amount of the said at least one oxidized lipid in the first sample to the amount in the second sample, whereby a change in the amount determined in the second sample in comparison to the first sample is indicative for neuropathic pain therapy being successful.

14. The method of claim 13, wherein said neuropathic pain therapy comprises administering a cytochrome P450 expoygenase (CYP)-antagonist.

15. (canceled)

16. The method of claim 3, wherein said at least one expoxylipid is 9,10-EpOME ((±)9(10)-epoxy-12Z-octadecaenoic acid).

17. The method of claim 9, wherein said epoxylipid is selected from the group consisting of: 9,10-EpOME ((±)9(10)-epoxy-12Z-octadecaenoic acid), 9-HODE ((±)9-hydroxy-10(E),12(Z)-octadecadienoic acid) and 13-HODE ((±)13-hydroxy-9(Z),11(E)-octadecadienoic acid).

18. The method of claim 9, wherein said at least one expoxylipid is 9,10-EpOME ((±)9(10)-epoxy-12Z-octadecaenoic acid).

19. The method of claim 10, wherein said chemotherapy-induced neuropathic pain (CIPN) is induced by paclitaxel and/or oxaliplatin.

20. The device of claim 12, wherein the at least one oxidized lipid as predictive and/or diagnostic biomarker is 9,10-EpOME.
